(19) United States
(12) Patent Application Publication
(10) Pub. No.: US 2013/0123254 A1
(43) Pub. Date: May 16, 2013

(54) PHARMACEUTICALLY ACCEPTABLE MGLUR5 POSITIVE ALLOSTERIC MODULATORS AND THEIR METHODS OF IDENTIFICATION

(71) Applicants: Barbara Biemans, Riehen (CH); Georg Jaeschke, Basel (CH); Lothar Lindemann, Basel (CH); Wolfgang Muster, Maulburg (DE); Heinz Stadler, Basel (CH); Eric Vieira, Frenkendorf (CH)

(72) Inventors: Barbara Biemans, Riehen (CH); Georg Jaeschke, Basel (CH); Lothar Lindemann, Basel (CH); Wolfgang Muster, Maulburg (DE); Heinz Stadler, Basel (CH); Eric Vieira, Frenkendorf (CH)

(21) Appl. No.: 13/616,619

(22) Filed: Sep. 14, 2012

(30) Foreign Application Priority Data
Sep. 30, 2011 (EP) 11183570.8

(51) Int. Cl. A61K 31/5377; A61K 31/501; A61K 31/444; A61K 31/5355; A61K 31/505; A61K 31/44; A61K 31/4545; A61K 31/4439; A61K 31/506

(52) U.S. Cl. CPC A61K 31/5377; A61K 31/4439; A61K 31/501; A61K 31/444; A61K 31/506; A61K 31/505; A61K 31/44; A61K 31/4545; A61K 31/5355
USPC 514/233.2; 546/276.1; 514/341; 546/273.7; 514/338; 544/238; 514/252.06; 546/268.7; 514/342; 546/271.4; 514/340; 546/256; 514/332; 544/332; 514/275; 544/335; 514/256; 546/323; 514/354; 546/194; 514/318; 544/96; 514/228.8; 544/118

(57) ABSTRACT

The present invention relates to mGluR5 positive allosteric modulators (PAM) and methods for identifying pharmaceutically acceptable compounds with high tolerability and safety, which method comprises the use of at least one non-competitive mGluR5 allosteric modulator which has a shift factor measured at 10 uM glutamate concentration below 3.0,

### PRIORITY TO RELATED APPLICATION(S)

This application claims the benefit of European Patent Application No. 1183570.8, filed Sep. 30, 2011, which is hereby incorporated by reference in its entirety.

### FIELD OF THE INVENTION

The positive allosteric modulation of mGluR5 is a valid approach to increase mGluR5 activity, and through the interaction of mGluR5 and NMDA may be able to normalize hypo-functional NMDA-mediated activity.

In the central nervous system (CNS) the transmission of stimuli takes place by the interaction of a neurotransmitter, which is sent out by a neuron, with a neuroreceptor.

Glutamate is the major excitatory neurotransmitter in the brain and plays a unique role in a variety of central nervous system (CNS) functions. The glutamate-dependent stimulus receptors are divided into two main groups. The first main group, namely the ionotropic receptors, forms ligand-controlled ion channels. The metabotropic glutamate receptors (mGluR) belong to the second main group and, furthermore, belong to the family of G-protein coupled receptors.

At present, eight different members of these mGluR are known and of these some even have sub-types. According to their sequence homology, signal transduction mechanisms and agonist selectivity, these eight receptors can be sub-divided into three sub-groups: mGluR1 and mGluR5 belong to group I, mGluR2 and mGluR3 belong to group II and mGluR4, mGluR6, mGluR7 and mGluR8 belong to group III.

Ligands of metabotropic glutamate receptors belonging to the first group can be used for the treatment or prevention of acute and/or chronic neurological disorders such as psychosis, epilepsy, schizophrenia, Alzheimer's disease, cognitive disorders and memory deficits, as well as chronic and acute pain.

Other treatable indications in this connection are restricted brain function caused by bypass operations or transplants, poor blood supply to the brain, spinal cord injuries, head injuries, hypoxia caused by pregnancy, cardiac arrest and hypoglycaemia. Further treatable indications are ischemia, Huntington's chorea, amyotrophic lateral sclerosis (ALS), dementia caused by AIDS, eye injuries, retinopathy, idiopathic parkinsonism or parkinsonism caused by medicaments as well as conditions which lead to glutamate-deficiency functions, such as e.g. muscle spasms, convulsions, migraine, urinary incontinence, nicotine addiction, opiate addiction, anxiety, vomiting, dyskinesia and depressions.

Disorders mediated full or in part by mGluR5 are for example acute, traumatic and chronic degenerative processes of the nervous system, such as Alzheimer's disease, senile dementia, Parkinson's disease, Huntington's chorea, amyotrophic lateral sclerosis and multiple sclerosis, psychiatric diseases such as schizophrenia and anxiety, depression, pain and drug dependency (Expert Opin. Ther. Patents (2002), 12, (12)).

A new avenue for developing selective modulators is to identify compounds which act through allosteric mechanism, modulating the receptor by binding to a site different from the highly conserved orthosteric binding site. Positive allosteric modulators of mGluR5 have emerged recently as novel pharmaceutical entities offering this attractive alternative. Positive allosteric modulators have been described, for example in WO2008/151184, WO2006/048771, WO2006/129199 and WO2005/044797 and in Molecular Pharmacology, 40, 333-336, 1991; The Journal of Pharmacology and Experimental Therapeutics, Vol 313, No. 1, 199-206, 2005;

Positive allosteric modulators do not directly activate receptors by themselves, but markedly potentiate agonist-stimulated responses, increasing the potency and maximum of efficacy of the agonist upon binding to the receptor. The binding of these compounds increases the affinity of a glutamate-site agonist at its extracellular N-terminal binding site. Allosteric modulation is thus an attractive mechanism for enhancing appropriate physiological receptor activation. There is a scarcity of selective positive allosteric modulators for the mGluR5 receptor. Conventional mGluR5 receptor modulators typically lack satisfactory aqueous solubility and exhibit poor oral bioavailability. Therefore, there remains a need for compounds that overcome these deficiencies and that effectively provide selective positive allosteric modulators for the mGluR5 receptor.

Positive allosteric modulators for the mGluR5 receptor are distinguished by having valuable therapeutic properties. They can be used in the treatment or prevention of disorders, relating to positive allosteric modulators for the mGluR5 receptor, which include schizophrenia, cognition, autism or tuberous sclerosis. (Curr. Drug Targets CNS Neurol. Disord., 2002, I, 261-281, Curr. Drug Targets CNS Neural. Disord., 2002, I, 1-19, Psychopharmacol. 2004, 174, 39-44).

### SUMMARY OF THE INVENTION

The present invention relates to mGluR5 positive allosteric modulators (PAM) and methods for identifying pharmaceutically acceptable compounds with high tolerability and safety, which method comprises the use of at least one non-competitive mGluR5 allosteric modulator which has a shift factor measured at 10 uM glutamate concentration below 3.0, preferably below 2.5. These identified mGluR5 positive allosteric modulators have an improved tolerability as well as safety, and can be used to treat diseases, disorders and conditions, which are related to the activity of mGluR5 positive allosteric modulators. These disorders and conditions include schizophrenia and other psychotic disorders, autism, tuberous sclerosis, and other conditions where an increase in mGluR5 tonus is desirable.

### DETAILED DESCRIPTION OF THE INVENTION

Surprisingly it has been found that mGluR5 positive allosteric modulators with high shift factors above 3 lead to severe CNS side effects, such as seizures and/or convulsions at doses which are close to those which show a therapeutic benefit (low therapeutic window). Therefore, there remains a need for identifying compounds which overcome these deficiencies, and which effectively provide selective positive allosteric modulators of the mGluR5 receptor with an appropriate pharmaceutical profile, which can be safely used as pharmaceuticals without undesirable and/or fatal CNS side effects. Therefore, the present invention relates to mGluR5 positive allosteric modulators with a shift factors below 3, which do not show CNS side effects while maintaining their desirable pharmaceutical activity. Furthermore, the present invention comprises a method to select mGluR5 positive allosteric modulators with desirable pharmacology and safety based upon measuring their shift factor at a given glutamate concentration, or using their measured efficacy at a given glutamate concentration as a surrogate marker for the shift factor; in order to select compounds devoid of undesirable CNS side effects at higher doses. Selection criteria are a shift factor below 3, preferably below 2.5 measured at 10 uM glutamate concentration, or an efficacy value below 70% preferably below 60%; measured at the EC₂₀ concentration of L-glutamate using the assay methods described below.

Other objects of the present invention are

mGluR5 positive allosteric modulators (PAM) with a shift factor below 3, measured at 10 uM,

mGluR5 positive allosteric modulators (PAM) with a shift factor below 3, wherein the compounds contain an acetylene (ethynyl) linker between two aryl or heteroaryl groups,

mGluR5 positive allosteric modulators (PAM) with a shift factor below 3 for use in predicting high pharmacological acceptance and

mGluR5 positive allosteric modulators (PAM), wherein the high pharmaceutical acceptance is related to improved tolerability and safety and to less un-desired side effects at higher doses.

A method for selecting mGluR5 positive allosteric modulators with high pharmacological acceptance comprising

measuring the shift factor of said mGluR5 positive allosteric modulators at a given glutamate concentration, or
measuring the efficacy data at a given glutamate concentration as a surrogate marker for the shift factor, and
treating a patient with this mGluR5 positive allosteric modulator if the shift factor is below 3.

A method for selecting mGluR5 positive allosteric modulators with high pharmacological acceptance wherein

the shift factor is below 3, measured at 10 uM glutamate concentration, or
the efficacy value is below 70%, measured at the EC₂₀ concentration of L-glutamate.

A method for selecting mGluR5 positive allosteric modulators with high pharmacological acceptance wherein the shift factor is below 2.5 and the efficacy value is below 60%.

A method for selecting mGluR5 positive allosteric modulators with high pharmacological acceptance wherein an intracellular Ca²⁺ mobilization assay is used.

A method for selecting mGluR5 positive allosteric modulators with high pharmacological acceptance comprising a dose-depending body temperature increase by administration of a pharmaco-logically active mGluR5 positive allosteric modulator.

### Biological Assay and Data

### Intracellular Ca²⁺ Mobilization Assay

A monoclonal HEK-293 cell line stably transfected with a cDNA encoding for the human mGlu5a receptor was generated; for the work with mGlu5 Positive Allosteric Modulators (PAMs), a cell line with low receptor expression levels and low constitutive receptor activity was selected to allow the differentiation of agonistic versus PAM activity. Cells were cultured according to standard protocols (Freshney, 2000) in Dulbecco's Modified Eagle Medium with high glucose supplemented with 1 mM glutamine, 10% (vol/vol) heat-inactivated bovine calf serum, Penicillin/Streptomycin, 50 μg/ml hygromycin and 15 μg/ml blasticidin (all cell culture reagents and antibiotics from Invitrogen, Basel, Switzerland).

About 24 hrs before an experiment, 5×10⁴ cells/well were seeded in poly-D-lysine coated, black/clear-bottomed 96-well plates. The cells were loaded with 2.5 μM Fluo-4AM in loading buffer (1×HBSS, 20 mM HEPES) for 1 hr at 37° C. and washed five times with loading buffer. The cells were transferred into a Functional Drug Screening System 7000 (Hamamatsu, Paris, France), and 11 half logarithmic serial dilutions of test compound at 37° C. were added and the cells were incubated for 10-30 min. with on-line recording of fluorescence. Following this pre-incubation step, the agonist L-glutamate was added to the cells at a concentration corresponding to EC₂₀ (typically around 80 μM) with on-line recording of fluorescence; in order to account for day-to-day variations in the responsiveness of cells, the EC₂₀ of glutamate was determined immediately ahead of each experiment by recording of a full dose-response curve of glutamate.

Responses were measured as peak increase in fluorescence minus basal (i.e. fluorescence without addition of L-glutamate), normalized to the maximal stimulatory effect obtained with saturating concentrations of L-glutamate. Graphs were plotted with the % maximal stimulatory using XLfit, a curve fitting program that iteratively plots the data using Levenburg Marquardt algorithm. The single site competition analysis equation used was y=A+((B−A)/(1+((x/C)D))), where y is the % maximal stimulatory effect, A is the minimum y, B is the maximum y, C is the EC₅₀, x is the log 10 of the concentration of the competing compound and D is the slope of the curve (the Hill Coefficient). From these curves the EC₅₀ (concentration at which half maximal stimulation was achieved), the Hill coefficient as well as the maximal response in % of the maximal stimulatory effect obtained with saturating concentrations of L-glutamate were calculated. This maximal stimulatory response corresponds to the efficacy value mentioned above (see FIG. 1).

Positive signals obtained during the pre-incubation with the PAM test compounds (i.e. before application of an EC₂₀ concentration of L-glutamate) were indicative of an agonistic activity, the absence of such signals were demonstrating the lack of agonistic activities. A depression of the signal observed after addition of the EC₂₀ concentration of L-glutamate was indicative of an inhibitory activity of the test compound. Shift factors were determined by recording the EC₅₀ dose-response curve of glutamate without test compound, as well as recording the EC₅₀ curve of glutamate on cells previously incubated for 10-30 min. with a fixed concentration of test compound (typically 5 μM and 10 μM), on the same plate. The shift factor is calculated for a specific concentration of the test compound according to the equation: Shift factor=EC₅₀(glutamate)/EC₅₀(glutamate+×uM test compound).

HEK293 cells stably expressing the mGlu5 receptor are prepared for the assay as described above. For the measurement, cells are incubated for 10-30 min. with a serial dilution of test compounds after which glutamate is applied at a concentration triggering a signal equivalent to an EC20. The peak fluorescence measured at each concentration of test compound is plotted as illustrated, and the PAM EC50 and efficacy are derived for each compound.

HEK293 cells stably expressing the mGlu5 receptor are prepared for the assay as described above. For the measurement, a serial dilution of test compounds is added to the cells. A Ca2+ mobilization signal without simultaneous or sequential addition of glutamate reveals agonistic activity of the mGlu5 PAM test compounds. The peak fluorescence measured at each concentration of test compound is plotted as illustrated, and the Agonism EC50 value is derived for each compound. Test compounds are typically probed for agonistic activity in the same assay run while measuring PAM EC50 activity by adding test compounds to the cells while recording fluorescence on-line (see FIG. 2).

HEK293 cells stably expressing the mGlu5 receptor are prepared for the assay as described above. On the same 96-well plate a serial dilution of glutamate is added to a) cells previously incubated for 10-30 min. with a fixed concentration of an mGlu5 PAM test compound and b) cells not incubated with mGlu5 PAM test compound. The peak fluorescence signals obtained from cells with or without pre-incubation with mGlu5 PAM test compound is plotted as illustrated, and the two EC50 values are derived from the plot. The shift factor is specific for the concentration of the test compound used and calculated as follows: Shift factor=ECM50(glutamate)/EC50(glutamate+×μM test compound).

In the list of examples below are shown the corresponding results for compounds which all have EC50<300 nM. (see FIG. 3).

Enclosed are typical experimental curves for Example 1 (low efficacy) and Example 15 (high efficacy), see FIGS. 4 and 5.

It can be seen from the two curves, both examples have an IC₅₀ around 10 nM but the efficacy (and shift factor) is lower for example 1 than for example 15.

Enclosed are also the corresponding shift factor curves for examples 1 and 15, see FIGS. 6 and 7.

It is seen from FIGS. 6 and 7, that the shift factor (concentration difference between curves A and C or A and D) is much higher for example 15 than for example 1.

### Pharmacological Assay and Data

### Body Temperature Test

Principle: Evaluation of the effect of a compound on core body temperature at a 2-h interval in the rat.

The temperature decrease in rats upon administration of mGluR5 negative allosteric modulators (antagonists) is well documented Warty & al.; Psychopharmacology (2005) 179: 207-217 (DOI 10.1007/s00213-005-2143-4)). We have found that administration of a pharmacologically active mGluR5 positive allosteric modulator produces a dose-dependent temperature increase in rats.

Male Sprague Dawley Rats (OFA-SD, Charles River, ˜250 g) are weighed and isolated for at least 30 min before compound injection in type II Makrolon cages. Before injection of the mGlu5 PAM compound, rectal body temperature is measured with a thermometer with a special rat probe for ˜20 s. The probe is dipped in glycerin before each use. Immediately after compound administration, rats are placed back in the type 2 cage. After 2 hours, the body temperature of each rat is measured again. The difference in absolute body temperature as well as the temperature difference delta T2-T0 between vehicle and compound injected rats is analysed.

This test has the advantages of being easy to perform, as well as giving very reliable and reproducible results. The pharmacological efficacies observed correlate very well will more sophisticated pharmacological models relevant for schizophrenia such as the Amphetamine induced hyperlocomotion test in rats which are much more difficult to perform.

The mGluR5 positive allosteric modulators (PAM) and pharmaceutically acceptable salts thereof can be used as medicaments, e.g. in the form of pharmaceutical preparations. The pharmaceutical preparations can be administered orally, e.g. in the form of tablets, coated tablets, dragées, hard and soft gelatine capsules, solutions, emulsions or suspensions. However, the administration can also be effected rectally, e.g. in the form of suppositories, or parenterally, e.g. in the form of injection solutions.

The compounds with the profile mentioned in claim 1 and pharmaceutically acceptable salts thereof can be processed with pharmaceutically inert, inorganic or organic carriers for the production of pharmaceutical preparations. Lactose, corn starch or derivatives thereof, talc, stearic acid or its salts and the like can be used, for example, as such carriers for tablets, coated tablets, dragées and hard gelatine capsules. Suitable carriers for soft gelatine capsules are, for example, vegetable oils, waxes, fats, semi-solid and liquid polyols and the like; depending on the nature of the active substance no carriers are, however, usually required in the case of soft gelatine capsules. Suitable carriers for the production of solutions and syrups are, for example, water, polyols, sucrose, invert sugar, glucose and the like. Adjuvants, such as alcohols, polyols, glycerol, vegetable oils and the like, can be used for aqueous injection solutions of water-soluble salts of compounds of formula (I), but as a rule are not necessary. Suitable carriers for suppositories are, for example, natural or hardened oils, waxes, fats, semi-liquid or liquid polyols and the like. In addition, the pharmaceutical preparations can contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances. As mentioned earlier, medicaments containing a compound of formula (I) or pharmaceutically acceptable salts thereof and a therapeutically inert excipient are also an object of the present invention, as is a process for the production of such medicaments which comprises bringing one or more compounds of formula I or pharmaceutically acceptable salts thereof and, if desired, one or more other therapeutically valuable substances into a galenical dosage form together with one or more therapeutically inert carriers.

As further mentioned earlier, the use of the compounds fulfilling the selection criteria mentioned in claim 1 and/or claim 2 for the preparation of medicaments useful in the prevention and/or the treatment of the above recited diseases is also an object of the present invention.

The dosage can vary within wide limits and will, of course, be fitted to the individual requirements in each particular case. In general, the effective dosage for oral or parenteral administration is between 0.01-20 mg/kg/day, with a dosage of 0.1-10 mg/kg/day being preferred for all of the indications described. The daily dosage for an adult human being weighing 70 kg accordingly lies between 0.7-1400 mg per day, preferably between 7 and 700 mg per day.

Tablets of the following composition are produced in a conventional manner:

| | mg/Tablet |
|---|---|
| Active ingredient | 100 |
| Powdered. lactose | 95 |
| White corn starch | 35 |
| Polyvinylpyrrolidone | 8 |
| Na carboxymethylstarch | 10 |
| Magnesium stearate | 2 |
| Tablet weight | 250 |

### LIST OF EXAMPLES AND DATA

The compounds described in the table are known, disclosed in EP11163708.8, PCT/EP2011/055585, EP11162945.7 or WO2011/073172.

| | | | | HTD/LTD | BT test |
|---|---|---|---|---|---|
| | | Shift | Shift | Dose (rat) | MED |
| | | (hum, | (rat, | [mg/Kg] | (rat) |
| Ex. | Structure | 10 uM) | 10 uM) | (observation) | [mg/Kg] |
| 1 | 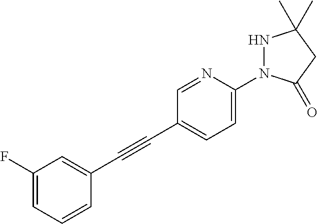 [SMILES: CC1(C)CC(=O)N(c2ccc(C#Cc3cccc(F)c3)cn2)N1] | 1.75 | 1.44 | 100 (well tolerated) | 0.3 |
| 2 | 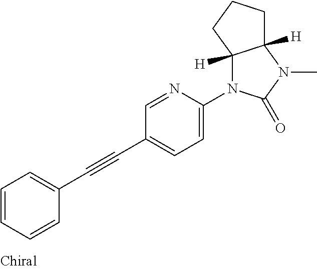 [SMILES: CN1C(=O)N(c2ccc(C#Cc3ccccc3)cn2)[C@@H]2CCC[C@@H]21] | 1.02 | 0.88 | 100 (well tolerated) | 0.1 |
| 3 | 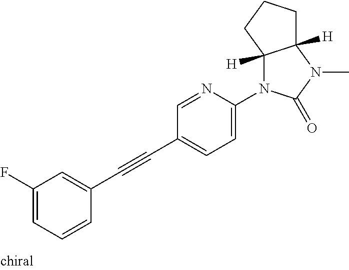 [SMILES: CN1C(=O)N(c2ccc(C#Cc3cccc(F)c3)cn2)[C@@H]2CCC[C@@H]21] | 0.87 | 0.82 | 100 (well tolerated) | 0.1 |
| 4 | 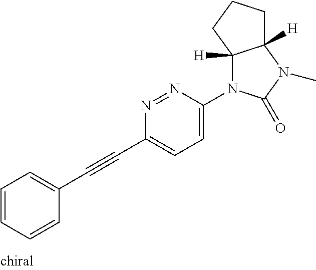 [SMILES: CN1C(=O)N(c2ccc(C#Cc3ccccc3)nn2)[C@@H]2CCC[C@@H]21] | 2.46 | 1.70 | 100 (well tolerated) | 0.1 |
| 5 | 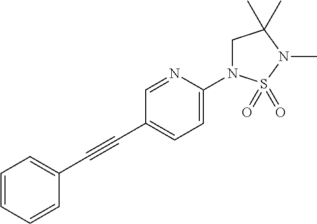 [SMILES: CN1C(C)(C)CN(c2ccc(C#Cc3ccccc3)cn2)S1(=O)=O] | 2.02 | 1.66 | 100 (well tolerated) | 0.3 |
| 6 | 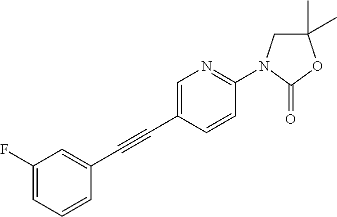 [SMILES: CC1(C)CN(c2ccc(C#Cc3cccc(F)c3)cn2)C(=O)O1] | 1.40 | 1.90 | 100 (well tolerated) | 0.3 |
| 7 | 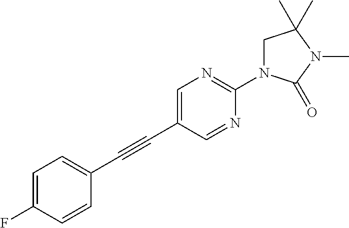 [SMILES: CN1C(=O)N(c2ncc(C#Cc3ccc(F)cc3)cn2)CC1(C)C] | 2.44 | 1.80 | 100 (well tolerated) | 0.3 |
| 8 | 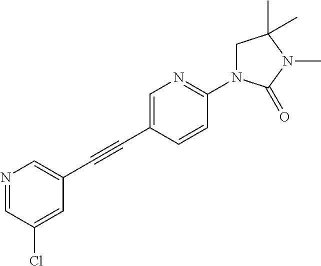 [SMILES: CN1C(=O)N(c2ccc(C#Cc3cncc(Cl)c3)cn2)CC1(C)C] | 3.60 | 2.28 | 100 (well tolerated) | 1 |
| 9 | 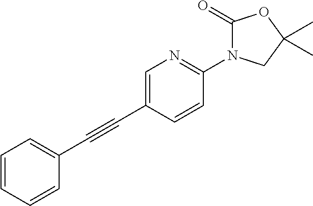 [SMILES: CC1(C)CN(c2ccc(C#Cc3ccccc3)cn2)C(=O)O1] | 1.70 | 2.63 | 30 (well tolerated) | 1 |
| 10 | 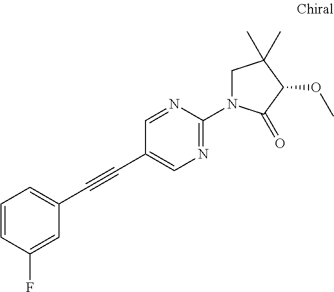 [SMILES: CO[C@@H]1C(=O)N(c2ncc(C#Cc3cccc(F)c3)cn2)CC1(C)C] | 3.46 | 2.62 | 100 (well tolerated) | 0.3 |
| 11 | 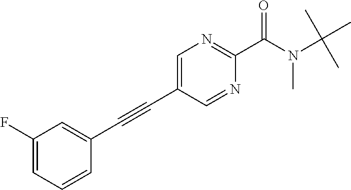 [SMILES: CN(C(=O)c1ncc(C#Cc2cccc(F)c2)cn1)C(C)(C)C] | 2.06 | 1.86 | 100 (well tolerated) | 0.1 |
| 12 | 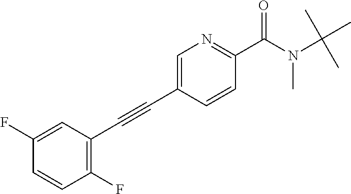 [SMILES: CN(C(=O)c1ccc(C#Cc2cc(F)ccc2F)cn1)C(C)(C)C] | 1.86 | 1.62 | 100 (well tolerated) | 0.1 |
| 13 | 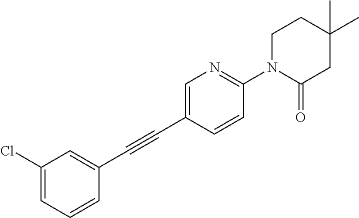 [SMILES: CC1(C)CCN(c2ccc(C#Cc3cccc(Cl)c3)cn2)C(=O)C1] | 4.74 | 3.92 | 100 (seizures) | 0.1 |
| 14 | 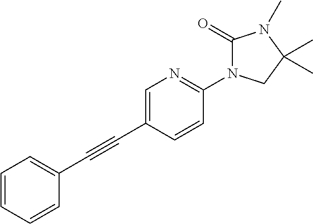 [SMILES: CN1C(=O)N(c2ccc(C#Cc3ccccc3)cn2)CC1(C)C] | 5.09 | 5.11 | 3 (seizures) | 0.3 |
| 15 | 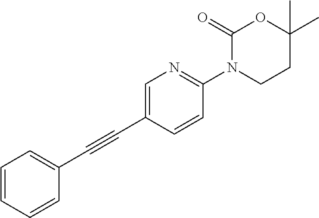 [SMILES: CC1(C)CCN(c2ccc(C#Cc3ccccc3)cn2)C(=O)O1] | 9.73 | 7.67 | 3 (seizures) | 0.1 |
| 16 | 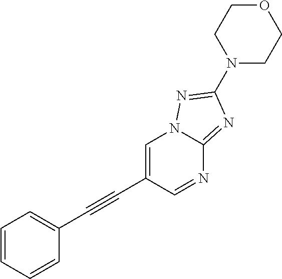 [SMILES: C(#Cc1cnc2nc(N3CCOCC3)nn2c1)c1ccccc1] | 12.13 | 11.31 | 10 (seizures) | 3 |
| 17 | 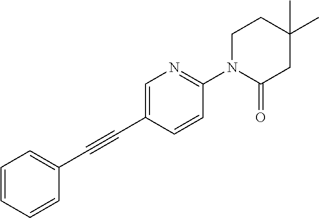 [SMILES: CC1(C)CCN(c2ccc(C#Cc3ccccc3)cn2)C(=O)C1] | 15.95 | 12.93 | 100 (seizures) | 0.1 |

From the table above can be clearly seen that all tested compounds with shift factors below 3 (ex. 1-12) are well tolerated at doses up to 30-100 mg/kg; whereas for compounds with higher shift factors (ex. 13-17) severe CNS side effects (seizures, convulsions) are observed at similar- and even sometimes at much lower doses. One can also observe, that although the shift factors are lower, the pharmacological in-vivo activities (minimal effective dose=MED) of the compounds in the body temperature test (BT) are similar. The compounds of the invention thus described are efficacious and well tolerated, even at high doses and do not lead to adverse CNS side effects.

### BRIEF DESCRIPTION OF THE DRAWINGS

FIG. 1:

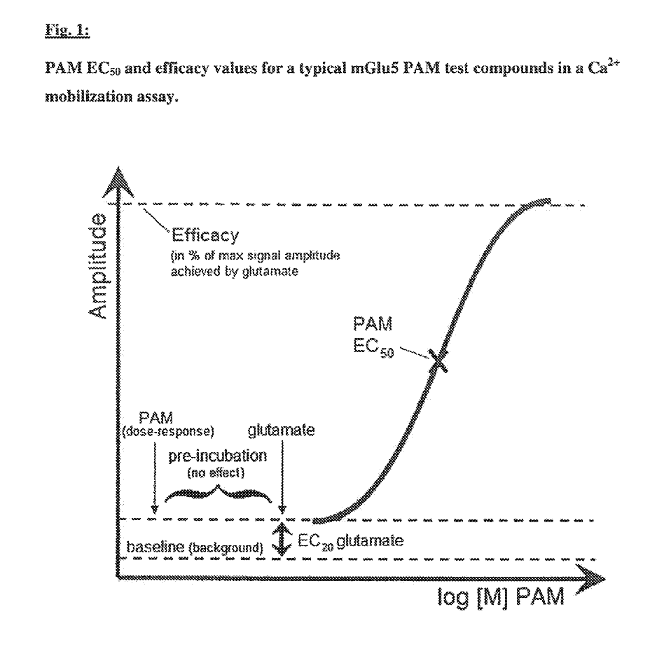

Measurement of PAM EC50 and efficacy values for a typical mGlu5 PAM test compounds in a Ca2+ mobilization assay.

FIG. 2:

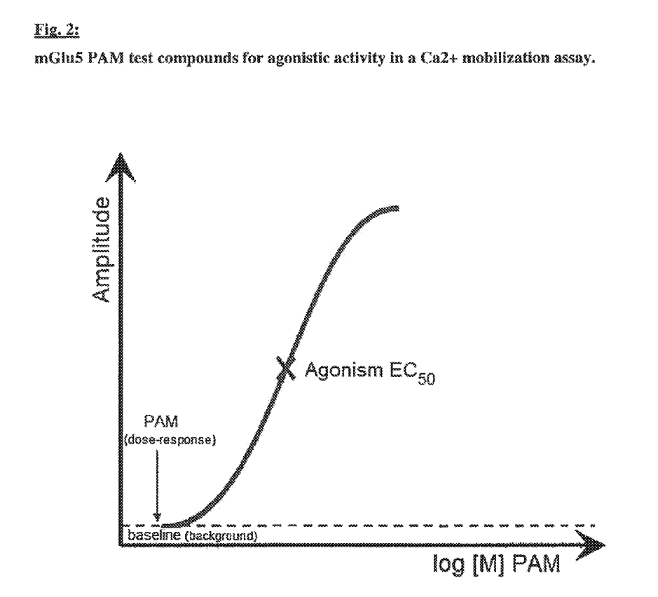

Testing mGlu5 PAM test compounds for agonistic activity in a Ca2+ mobilization assay.

FIG. 3:

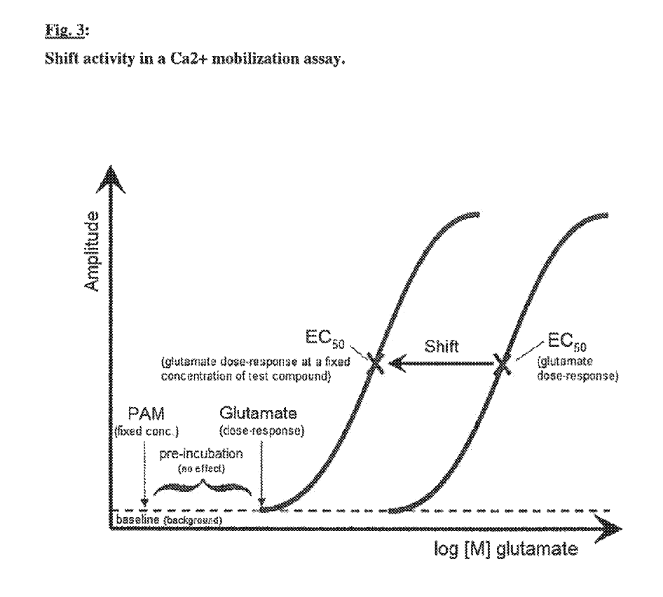

Measuring Shift activity in a Ca2+ mobilization assay.

FIG. 4

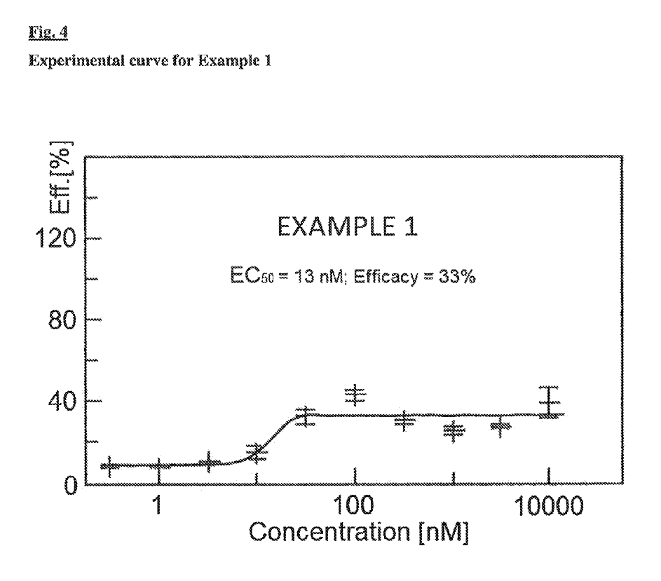

Experimental curves for Example 1

FIG. 5

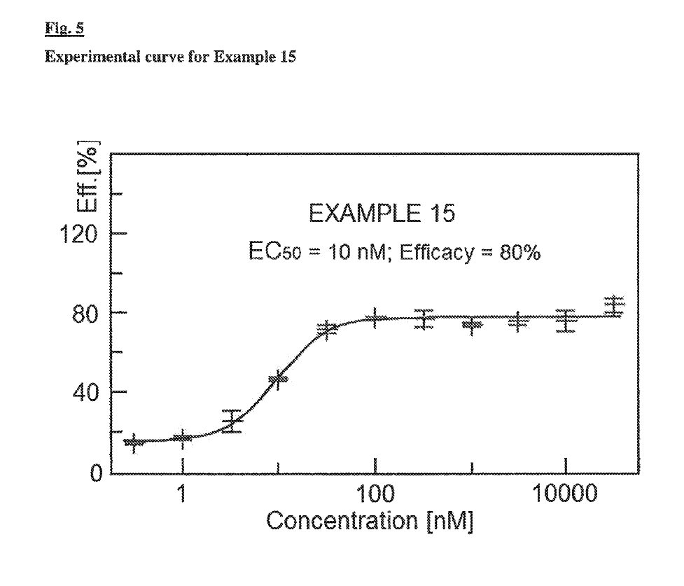

Experimental curves for Example 15

FIG. 6

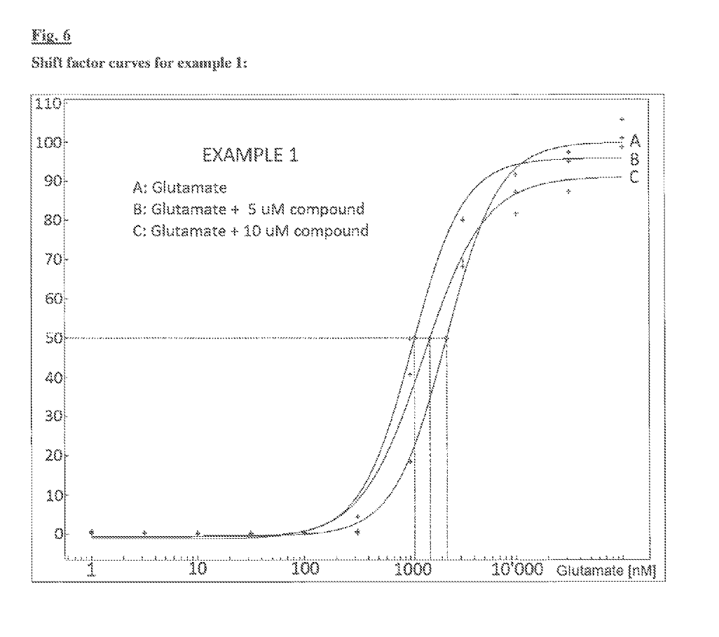

Shift factor curves for examples 1

FIG. 7

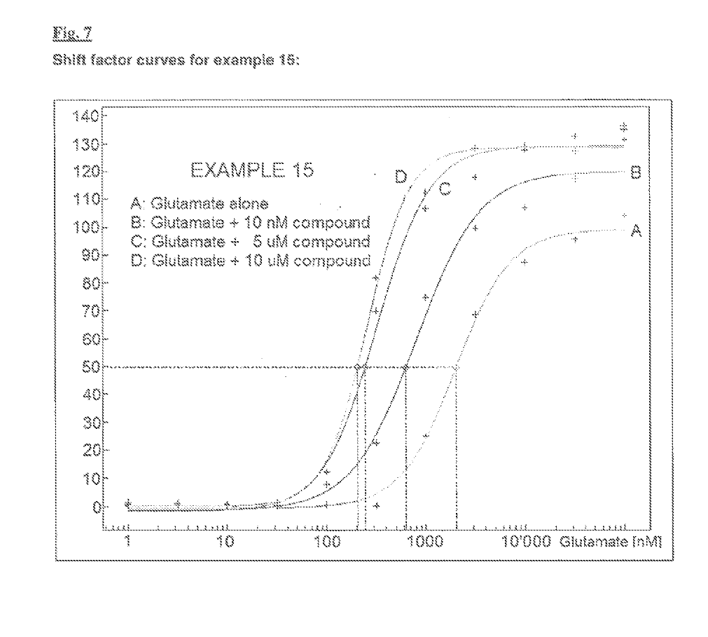

Shift factor curves for examples 15

FIG. 8:

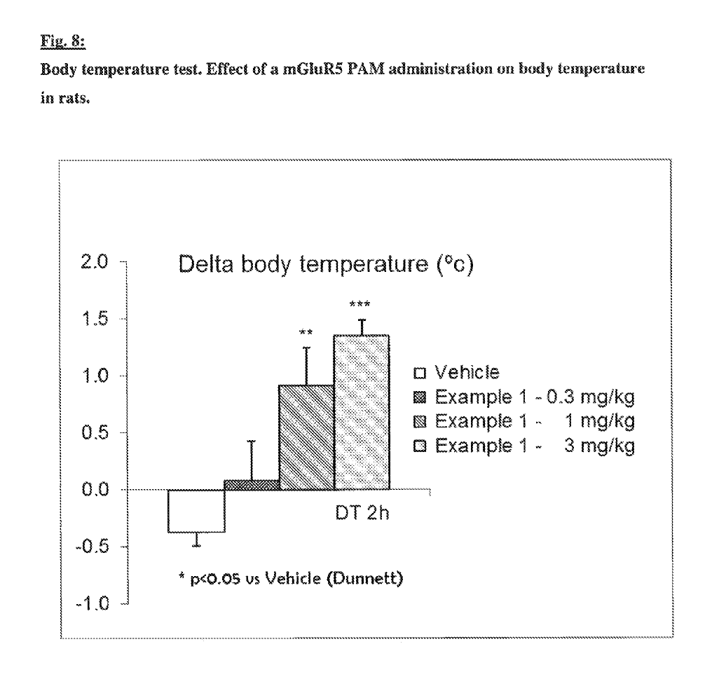

Body temperature test. Effect of a mGluR5 PAM administration on body temperature in rats.

What is claimed:

1. mGluR5 positive allosteric modulators (PAM) with a shift factor below 3 at 10 uM glutamate concentration.

2. The mGluR5 positive allosteric modulators (PAM) of claim 1, wherein the compounds contain an ethylene linker between two aryl or heteroaryl groups.

3. The mGluR5 positive allosteric modulators (PAM) of claim 2 for use in predicting high pharmacological acceptance.

4. The mGluR5 positive allosteric modulators (PAM) of claim 3, wherein the high pharmaceutical acceptance is related to improved tolerability and safety and to less un-desired side effects at higher doses.

5. A method for selecting mGluR5 positive allosteric modulators with high pharmacological acceptance comprising
measuring the shift factor of said mGluR5 positive allosteric modulators at a given glutamate concentration, or
measuring the efficacy data at a given glutamate concentration as a surrogate marker for the shift factor and
treating a patient with this mGluR5 positive allosteric modulator if the shift factor is below 3.

6. A method for selecting mGluR5 positive allosteric modulators with high pharmacological acceptance according to claim 5, wherein
the shift factor is below 3, measured at 10 uM glutamate concentration, or
the efficacy value is below 70%, measured at the EC₂₀ concentration of L-glutamate.

7. A method for selecting mGluR5 positive allosteric modulators with high pharmacological acceptance according to claim 6, wherein the shift factor is below 2, 5 and the efficacy value is below 60%.

8. A method for selecting mGluR5 positive allosteric modulators with high pharmacological acceptance according to claim 7, wherein an intracellular Ca²⁺ mobilization assay is used.

9. A method for selecting mGluR5 positive allosteric modulators with high pharmacological acceptance according to claim 8, comprising a dose-depending body temperature increase by administration of a pharmacologically active mGluR5 positive allosteric modulator.
